(19) United States
(12) Patent Application Publication
(10) Pub. No.: US 2005/0004181 A1
(43) Pub. Date: Jan. 6, 2005

(54) Use of anti-emetic for pre and post operative care

(75) Inventor: Brenda Reeve, Ancaster (CA)

Correspondence Address: Michael R. Krawzsenek;Fulbright & Jaworski L.L.P., Suite 2400, 600 Congress Avenue, Austin, TX, 78701, US

(73) Assignee: Duchesnay Inc., Laval (CA)

(21) Appl. No.: 10/613,753

(22) Filed: Jul. 3, 2003

(51) Int. Cl. A61K 31/4415; A61K 31/44

(52) U.S. Cl. 514/350; 514/357

(57) ABSTRACT

A method for the prophylaxis and treatment of post-surgical vomiting comprising administering to a patient a therapeutically effective amount of Doxylamine Succinate and Pyridoxine Hydrochloride. The method can also include pre-operative, peri-operative and/or post-operative administration of Doxylamine Succinate and Pyridoxine Hydrochloride to a patient.

### FIELD OF THE INVENTION

The present invention relates to a method and pharmaceutical compositions for the prophylaxis and treatment of post-surgical vomiting. More specifically, the present invention relates to a newly discovered indication for a known drug. The drug comprises Doxylamine Succinate and Pyridoxine Hydrochloride (vitamin B6). This drug is currently marketed in Canada under the registered trademark Diclectin®.

### BACKGROUND OF THE INVENTION

Post-operative vomiting is an important problem and from a patient standpoint, one of the most commonly reported and distressing post-operative complication. Patients commonly report post-operative vomiting as a greater source of discomfort than pain.

From a clinical standpoint, post-operative vomiting is troublesome and requires the presence of clinical staff to ensure that patients do not choke or otherwise harm themselves. In many surgical interventions it is clinically important that patients do not vomit and cause strain on stitches. Ruptured stitches, especially when stitches are internal, can lead to hemorrhage, which can in turn lead to further surgery and in general terms leads to a setback in patient recovery.

Thus from the standpoint of both patients and clinicians, the control of post-operative vomiting is essential.

Financially, the control of post-operative vomiting is also important. Industrialized society outpatient surgery is common and the importance of being able to send patients home without an overnight stay is financially attractive. Thousands of day surgeries are performed everyday in most countries of the world. Thus, millions of such operations are completed each year. As a societal cost, post-operative vomiting slows recovery, return to productive activities and uses up valuable health care resources.

Many anesthetists currently use prophylactic anti-vomiting drugs such as metoclopramide, chloropromazine, diphenhydramine, dimenhydrinate, meclizine, cyclizine before or during surgery. However, it is common not to use anti-vomiting drugs at all due to either poor efficacy of current agents or troublesome side-effects such as dystonic reactions and somnolence.

Since the 1950's it has been known to use the drug now sold as Diclectin® as an anti-nauseant and anti-emetic for the treatment of hyperemesis gravidarum among pregnant women (Brent, 1983).

Hyperemesis gravidarum is an extreme form of nausea and vomiting encountered during pregnancy. Up to now, there has been a belief that Diclectin® was effective because it would safely control symptoms of the hormonal and physiological upheaval causing nausea and vomiting during pregnancy. Indeed, systematic and randomized clinical trials have consistently demonstrated the safety and efficacy of Diclectin® in a pregnancy setting (Jewell. 1993).

It has also previously been shown that Diclectin® may be efficacious in curbing nausea in terminal disease situations when hormonal and physiological functions are greatly disrupted by disease and side effects caused by potent drugs such as anti-cancer drugs (Canadian Patent 2,139,896).

However, it has been unknown and unproven that Diclectin® could be used as an anti-emetic in a general population setting undergoing standard surgical interventions. Such discovery is of great practical significance considering the large number of such surgical interventions and the fact that post-surgical vomiting is an important drawback to rapid recovery.

In light of the foregoing, there remains a constant need for new post-operative anti-vomiting drugs which are safe, efficacious and which exhibit few or mild side-effects.

### SUMMARY OF THE INVENTION

The inventors have overcome the deficiencies of the prior art by providing a method for the prophylaxis and treatment of post-surgical vomiting. In one aspect of the present invention, there is provided a method of reducing post-surgical vomiting comprising administering to a patient a therapeutically effective amount of Doxylamine Succinate and Pyridoxine Hydrochloride. The Doxylamine Succinate and Pyridoxine Hydrochloride can be administered before, during and/or after surgery. In particular embodiments, the Doxylamine Succinate and Pyridoxine Hydrochloride can be administered after surgery at regular intervals. Doxylamine Succinate and Pyridoxine Hydrochloride can also be administered before and/or during surgery but not after surgery.

In certain aspects of this invention, Doxylamine Succinate and Pyridoxine Hydrochloride can be administered orally. When Doxylamine Succinate and Pyridoxine Hydrochloride are administered orally, they can be formulated in a delayed release formulation. The orally delayed release formulation can be enterically coated.

In another embodiment, Doxylamine Succinate and the Pyridoxine Hydrochloride can be formulated in a pharmaceutically acceptable carrier.

In specific aspects of this invention, at least about 10 mg of Doxylamine Succinate and at least about 10 mg of Pyridoxine Hydrochloride can be administered to the patient to reduce, prevent or treat post-surgical vomiting. In other embodiment, at least about 20 mg of Doxylamine Succinate and at least about 20 mg of Pyridoxine Hydrochloride can be given to a patient.

In particular embodiments, the patient is a woman. The surgery can be performed on an outpatient basis.

In still another aspect of this invention, Doxylamine Succinate and Pyridoxine Hydrochloride can be administered before anesthesia is administered to the patient. In other embodiments, Doxylamine Succinate and Pyridoxine Hydrochloride can be administered on an evening prior to surgery, a morning of the day of surgery and/or immediately after surgery.

Doxylamine Succinate can be administered before, at substantially the same time or after Pyridoxine Hydrochloride is administered to the patient.

In more specific aspects of this invention, there is provided a method of treating, preventing or reducing post-surgical vomiting comprising pre-operative and or peri-operative administration of a therapeutically effective amount of Doxylamine Succinate and Pyridoxine Hydrochloride. In one embodiment, the Doxylamine Succinate and Pyridoxine Hydrochloride are administered by administering to the patient Diclectin®. Diclectin® is an anti-vomiting drug that is currently marketed in Canada. Optionally, the method also includes the further step of post-operative administration of the same drug. In a preferred embodiment, Diclectin® will be administered pre and/or peri-operatively such as prior to induction of anesthesia or concurrently with anesthesia and/or postoperatively.

A “patient” or “subject,” as used herein, may be an animal. Preferred animals are mammals, including but not limited to humans, pigs, cats, dogs, rodents, horses, cattle, sheep, goats and cows. Preferred patients and subjects are humans.

“Reducing post-surgical vomiting” means any measurable decrease of post surgical-vomiting. Similarly, “reducing” means any measurable decrease or complete inhibition of post-surgical vomiting.

The words “a” and “an,” as used in this specification, including the claims, denotes “one or more.” Specifically, the use of “comprising,” “having,” or other open language in claims that claim a combination or method employing “an object,” denotes that “one or more of the object” may be employed in the claimed method or combination.

Other objects, advantages and features of the present invention will become more apparent upon reading the following non-restrictive description of preferred embodiments included throughout this specification and the example provided below.

### DESCRIPTION OF THE PREFERRED EMBODIMENT

A. Diclectin®

Diclectin® is an anti-nauseant approved for use during pregnancy. It is currently sold in Canada in delayed release formulation containing 10 mg of Doxylamine Succinate (an antihistamine) and 10 mg of Pyridoxine Hydrochloride (Vitamin B6).

Doxylamine Succinate has the following chemical formula:

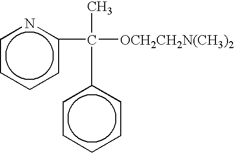

Pyridoxine Hydrochloride has the following chemical formula:

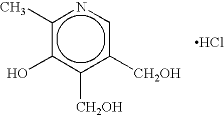

It has now been shown, against prior art expectations that pre, and/or peri-operative and/or post-operative use of Diclectin® reduces the incidence of post-operative vomiting in patients undergoing surgery such as elective laparoscopic tubal ligation. This newly discovered indication can be described as the use of Diclectin® in the prophylaxis and treatment of post-operative vomiting.

2. Combination Therapy

In order to increase the effectiveness of the present invention, it may be desirable to combine the administration Doxylamine Succinate and Pyridoxine Hydrochloride with other known drugs, compounds, agents, and methods effective in the preventing or reducing vomiting, including post-surgical vomiting. Drugs that may be used in combination with the present invention include, but are not limited to, metoclopramide, chloropromazine, diphenhydramine, dimenhydrinate, meclizine and cyclizine.

This process may involve administering the combination of Doxylamine Succinate and Pyridoxine Hydrochloride with other agent(s) to the patient at the same time, for example, using a single composition or pharmacological formulation that includes Doxylamine Succinate, Pyridoxine Hydrochloride and an additional anti-vomiting agent or drug. In other embodiments of this invention, the combination can be separated into two distinct compositions or formulations given at the same time, wherein one composition includes Doxylamine Succinate and Pyridoxine Hydrochloride and the second composition includes a known anti-vomiting agent or agents. The second agent therapy may precede or follow the administration of Doxylamine Succinate and Pyridoxine Hydrochloride to the patient by intervals ranging from minutes to weeks.

The exact schedule of treatment with Doxylamine Succinate and Pyridoxine Hydrochloride combination therapy and the second agent is determined in large part by the pharmacokinetic or pharmacodynamic properties of Doxylamine Succinate and Pyridoxine Hydrochloride and the second agents.

In embodiments where the other agent and Doxylamine Succinate and Pyridoxine Hydrochloride combination therapy are administered separately to the subject, one may wish that a significant period of time does not expire between the time of each delivery, such that the second agent and the Doxylamine Succinate and Pyridoxine Hydrochloride combination therapy would be able to exert an advantageously combined effect on the subject. In such instances, it is contemplated that one may administer to the subject with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, the Doxylamine Succinate and Pyridoxine Hydrochloride combination therapy is “A” and the second agent is “B”:
A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A
   3. Formulations and Routes of Administrations

Pharmaceutical compositions of the present invention include Doxylamine Succinate and Pyridoxine Hydrochloride. The phrases “pharmaceutical or pharmacologically acceptable” refers to molecular entities and compositions that do not produce an adverse, allergic or other untoward reaction when administered to an animal, such as, for example, a human. The preparation of a pharmaceutical composition comprising Doxylamine Succinate and Pyridoxine Hydrochloride will be known to those of skill in the art in light of the present disclosure, as exemplified by Remington's Pharmaceutical Sciences, 18th Ed. Mack Printing Company, 1990. Moreover, for animal (e.g., human) administration, it will be understood that preparations should meet sterility, pyrogenicity, general safety and purity standards as required by FDA Office of Biological Standards.

“Therapeutically effective amounts” are those amounts effective to produce beneficial results, particularly with respect to treating post-surgical vomiting in a patient. Such amounts may be initially determined by reviewing the published literature, by conducting in vitro tests or by conducting metabolic studies in healthy experimental animals.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Except insofar as any conventional carrier is incompatible with the active ingredient, its use in the therapeutic or pharmaceutical compositions is contemplated.

The compositions of the present invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (Remington's, 1990).

The actual dosage amount of a composition of the present invention administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The compositions may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In certain embodiments, the compositions are prepared for administration by such routes as oral ingestion. In these embodiments, the solid composition may comprise, for example, solutions, suspensions, emulsions, tablets, pills, capsules (e.g., hard or soft shelled gelatin capsules), sustained time release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments, an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments, the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition should be stable under the conditions of manufacture and storage, and preserved against the contaminating action of microorganisms, such as bacteria and fungi. It will be appreciated that exotoxin contamination should be kept minimally at a safe level, for example, less that 0.5 ng/mg protein.

### EXAMPLES

The following example is included to demonstrate new and inventive methods of the inventor and preferred embodiments of the invention. It should be appreciated by those of skill in the art that the techniques disclosed in the example which follows represent techniques discovered by the inventor to function well in the practice of the invention and, thus, can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Materials and Methods: The present invention is evidence in a randomized, stratified, double-blind, placebo-controlled study as described in the following paragraphs. In the study, a total of 146 patients were recruited. Of the 146 patients enrolled in the study, 14 patients cancelled surgery. Others were removed from the study because of unavailable data, withdrawn consent or break in protocol. Thus, out of 146 patients, the data from 102 patients was considered.

In the study, 102 women who underwent day surgery (tubal ligation), Diclectin® or placebo were administered in a randomized, stratified, double-blind manner. Patients were stratified according to the timing of their menstrual cycle such that at least 30% of the patients were in day 0-8 of their menstrual cycle. In each case, patients were given, either a placebo or Diclectin® dosage units, each unit containing about 10 mg of Doxylamine Succinate (an antihistamine) and about 10 mg of Pyridoxine Hydrochloride (Vitamin B6) at specified time intervals. More specifically, patients were instructed to take 2 pills with fluid before retiring to bed on the night prior to surgery. Upon awakening the morning of surgery, they took 1 more tablet with a sip of water, and any other pre-operative medications as instructed by their anesthesiologist (no pre-operative anti-emetics). The fourth tablet was taken post-operatively, before discharge form the short stay unit.

For the tubal ligation, patients received a standard anesthetic with no pre-operative anti-emetics. Induction of anesthesia consisted of propofol (1-3 mg/kg) plus an opioid dose equivalent to fentanyl 102 ug/kg. Muscle relaxation was achieved with rocuronium (0.6 mg/kg) or vecuronium (0.1 mg/kg) at the discretion of the attending anesthesiologist. Anesthesia was maintained with nitrous oxide (70%), oxygen (30%), and isoflurane (0.5-1.5%). Patients received volumed cycled mechanical ventilation. Muscle relaxation was monitored by peripheral nerve stimulator and, at the end of surgery, was reversed with neostigmine (50 ug/kg) and glycopyrrolate (10 ug/kg). Patients were transferred post-operatively to the recovery room, and transferred to the short stay unit until ready for discharge home.

Post-operatively, there were 3 periods of observation in different venues. The first observation period was in the Post Anesthetic Care Unit (PACU) and outcomes were recorded by the PACU nurse.

The second observation period was in the Short Stay Unit (SSU), and outcomes were recorded by the SSU nurse. Those 2 periods covered the time from 0-6 hours.

The third period spanned the time from hospital discharge until 24 hours postoperatively (6-24 hours); data were collected for this period using interviewer-administered telephone follow-up by the principal investigator.

The outcomes were measured during each of the 3 post-operative observation periods included incidence of nausea, incidence of vomiting, possible side-effects including headache, epigastric discomfort, dizziness, or drowsiness, and administration of post-operative rescue anti-emetic drugs.

Patient symptoms of nausea and vomiting were measured separately in this trial. Nausea was recorded as absent (0), mild (1), moderate (2), or severe (3). Vomiting was recorded as absent (0), vomited once (1), vomited more than once (2). A priori, it was determined that a difference in one point would represent a clinically important difference to patients.

For statistical calculations Chi square analysis was used to compare the proportions of patients in each group with these symptoms. Two sided significance test was used and p<0.05 was set as the barrier indicating complete statistical significance.

Results: The results are shown in Table 1 below:

TABLE 1

| | | Allocation | | |
|---|---|---|---|---|
| | | DICLECTIN | | |
| TIME | | ® | PLACEBO | |
| FRAME | OUTCOME | N = 50 | N = 52 | p-value |
| PACU | Nausea | 12/50 (24%) | 10/51 (19.6%) | 0.593 |
| | Vomiting | 2/50 (4%) | 4/51 (7.8%) | 0.678 |
| | ≧1 dose of anti- | 10/50 (20%) | 10/51 (19.6%) | 0.885 |
| | emetic given | | | |
| SSU | Nausea | 20/50 (40%) | 16/51 (31.4%) | 0.365 |
| | Vomiting | 9/49 (18.4%) | 9/52 (17.3%) | 0.889 |
| | ≧1 dose of anti- | 7/50 (14%) | 10/52 (19.2%) | 0.479 |
| | emetic given | | | |
| 6-24 | Nausea | 14/50 (28%) | 15/48 (31.3%) | 0.725 |
| HRS. | Vomiting | 5/50 (10%) | 12/48 (25%) | 0.04995 |
| | ≧1 dose of anti- | 1/50 (2%) | 2/48 (4.2%) | 0.292 |
| | emetic given | | | |
| Functional | Return to usual | 1.510 | 4.065 | 0.098 |
| Outcome | activities (days) | | | |

Significance of Results: In this randomized double-blind placebo-controlled trial, it was clearly demonstrated that Diclectin® significantly reduced the incidence of post-operative vomiting.

As an effective agent for preventing post-operative vomiting, Diclectin® is an attractive alternative to medications currently given either prophylactically or to treat vomiting in post-op recovery areas.

Diclectin® is preferably provided as an oral delayed release formulation. Because it does not require an injection, Diclectin® can be easily ingested by patients pre-operatively and after discharge from hospital.

Known anti-emetics given intravenously may be efficacious in preventing in-hospital nausea and vomiting but will have worn off by the time patients are home. In the study reported above in Table 1, it was shown that Diclectin® significantly reduced the occurrence of post-operative vomiting even after hospital discharge during the first 24 hours at home.

Also shown in Table 1 is the trend of an accelerated return to normal activities and work associated with Diclectin®. This is also a significant finding. A return to work after 1.5 days on Diclectin® is a strong benefit in comparison to 4.1 days for patients on placebo.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention.

### REFERENCES

The following references, to the extent that they provide exemplary procedural or other details supplementary to those set forth herein, are specifically incorporated herein by reference.
Brent, Teratology, 27:283-286, 1983.
Canadian Patent 2,139,896
Jewell, In: Pregnancy and childbirth module, Enkin et al. (Eds.), Disk issue 1, Cochrane Database of Systematic Reviews, Review No 03351, Oxford, 1993.
Remington's Pharmaceutical Sciences, 18th Ed. Mack Printing Company, 1990.

1. A method of reducing post-surgical vomiting comprising administering to a patient a therapeutically effective amount of Doxylamine Succinate and Pyridoxine Hydrochloride.

2. The method of claim 1, wherein Doxylamine Succinate and Pyridoxine Hydrochloride are administered before surgery.

3. The method of claim 1, wherein Doxylamine Succinate and Pyridoxine Hydrochloride are administered during surgery.

4. The method of claim 1, wherein Doxylamine Succinate and Pyridoxine Hydrochloride are administered after surgery.

5. The method of claim 4, wherein Doxylamine Succinate and Pyridoxine Hydrochloride are administered after surgery at regular intervals.

6. The method of claim 1, wherein Doxylamine Succinate and Pyridoxine Hydrochloride are administered before, during and after surgery.

7. The method of claim 1, wherein Doxylamine Succinate and Pyridoxine Hydrochloride are administered orally.

8. The method of claim 7, wherein Doxylamine Succinate and Pyridoxine Hydrochloride are administered orally in a delayed release formulation.

9. The method of claim 8, wherein the orally delayed release formulation is enterically coated.

10. The method of claim 1, wherein Doxylamine Succinate and Pyridoxine Hydrochloride are formulated in a pharmaceutically acceptable carrier.

11. The method of claim 1, further defined as administering at least about 10 mg of Doxylamine Succinate and at least about 10 mg of Pyridoxine Hydrochloride.

12. The method of claim 1, further defined as administering at least about 20 mg of Doxylamine Succinate and at least about 20 mg of Pyridoxine Hydrochloride.

13. The method of claim 1, wherein Doxylamine Succinate and Pyridoxine Hydrochloride are administered before anesthesia is administered to the patient.

14. The method of claim 1, wherein the patient is a woman.

15. The method of claim 1, wherein the surgery is performed on an outpatient basis.

16. The method of claim 1, wherein Doxylamine Succinate and Pyridoxine Hydrochloride are administered on an evening prior to surgery, a morning of the day of surgery or immediately after surgery.

17. The method of claim 1, wherein Doxylamine Succinate is administered before, at substantially the same time or after Pyridoxine Hydrochloride is administered to the patient.

18. A method of treating post-surgical vomiting comprising administering to a patient a therapeutically effective amount of Doxylamine Succinate and Pyridoxine Hydrochloride before, during or after surgery.

19. The method of claim 18, wherein Doxylamine Succinate and Pyridoxine Hydrochloride are administered before, during and after surgery.

20. The method of claim 18, wherein Doxylamine Succinate and Pyridoxine Hydrochloride are administered orally in a delayed release formulation.

21. The method of claim 18, wherein Doxylamine Succinate is administered before, at substantially the same time or after Pyridoxine Hydrochloride is administered to the patient.

22. The method of claim 18, wherein Doxylamine Succinate and Pyridoxine Hydrochloride are formulated in a pharmaceutically acceptable carrier.

23. The method of claim 18, further defined as administering at least about 10 mg of Doxylamine Succinate and at least about 10 mg of Pyridoxine Hydrochloride.
